(19) United States
(12) Patent Application Publication
(10) Pub. No.: US 2019/0300483 A1
(43) Pub. Date: Oct. 3, 2019

(54) POLYMORPHS OF BETRlXABAN & ITS MALEATE SALT

(71) Applicant: DR. REDDY'S LABORATORIES LIMITED, Hyderabad (IN)

(72) Inventors: Subba Reddy PEDDI REDDY, Hyderabad (IN); Venkata Narasayya SALADI, Hyderabad (IN); Vishweshwar PEDDY, Hyderabad (IN); Satarupa BHATTACHARJEE, Howrah (IN)

(73) Assignee: Dr. Reddy's Laboratories Limited, Hyderabad (IN)

(21) Appl. No.: 16/306,467

(22) PCT Filed: May 31, 2017
(86) PCT No.: PCT/IB2017/053201

§ 371 Date: Nov. 30, 2018

(30) Foreign Application Priority Data
Jun. 2, 2016 (IN) 201641019078

(51) Int. Cl. C07D 213/75

(52) U.S. Cl. CPC C07D 213/75; C07B 2200/13

(57) ABSTRACT

The present application relates to solid state forms of Betrixaban and its Maleate salt, and processes for preparation thereof.

### FIELD OF THE APPLICATION

The present application relates to solid state forms of Betrixaban, and its maleate salt and processes for preparation thereof.

The drug compound having the adopted name “Betrixaban” and it has chemical name: N-(5-chloropyridin-2-yl)-2-(4-(N,N-dimethylcarbamimidoyl)benzamido)-5-methoxy benzamide; and a structure depicted by Formula I.

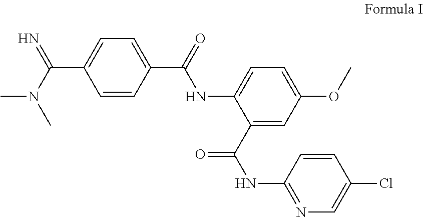 [SMILES: COc1ccc(NC(=O)c2ccc(C(=N)N(C)C)cc2)c(C(=O)Nc2ccc(Cl)cn2)c1]

International Patent Application Publication Nos. WO 2001019788A2, WO 2001064643A2, WO2001064642A3 which are incorporated herein in their entirety reported Betrixaban and its related compounds as a potent FXa inhibitor. Such compounds inter alia can be used for the potential extended-duration prophylaxis and treatment of venous thrombosis in acute medically ill patients. International Patent Application Publication Nos. WO 2008/057972A1 and WO 2011/084519A1 disclose processes for preparing Betrixaban and its salts.

International Patent Application Publication No. WO 2007/056517A2 and WO 2012/031017A1 disclose crystalline forms of the compound of Formula (I).

Polymorphism, the occurrence of different crystal forms, is a property of some molecules and molecular complexes. A single molecule, like Betrixaban maleate, may give rise to a variety of crystalline forms having distinct crystal structures and physical properties like melting point, X-ray diffraction pattern, infrared absorption fingerprint, and solid state NMR spectrum. One crystalline form may give rise to thermal behavior different from that of another crystalline form.

The difference in the physical properties of different crystalline forms results from the orientation and intermolecular interactions of adjacent molecules or complexes in the bulk solid. Accordingly, polymorphs are distinct solids sharing the same molecular formula yet having advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubility.

The discovery of new polymorphic forms or solvates of a pharmaceutically useful compound provides a new opportunity to improve the performance characteristics of a pharmaceutical product. It enlarges the repertoire of materials that a formulation scientists has available for designing, for example, a pharmaceutical dosage form of a drug with a targeted release profile or other desired characteristics. Therefore, there is a need for additional crystalline forms of Betrixaban maleate.

Since improved drug formulations are consistently sought, there is an ongoing need for new or purer polymorphic form of existing drug molecules. The present invention describes polymorphs of Betrixaban maleate that helps to meet aforementioned and other needs.

The present invention provides novel crystalline forms of Betrixaban maleate.

Crystalline solids normally require a significant amount of energy for dissolution due to their highly organized, lattice like structures. For example, the energy required for a drug molecule to escape from a crystal is more than from an amorphous or a non-crystalline form. It is known that the amorphous forms in a number of drugs exhibit different dissolution characteristics and in some cases different bioavailability patterns compared to the crystalline form. For some therapeutic indications, one bioavailability pattern may be favored over another. Therefore, it is desirable to have amorphous forms of drugs with high purity to meet the needs of regulatory agencies and also reproducible processes for their preparation.

In view of the above, it is therefore, desirable to have stable amorphous form of Betrixaban and its maleate salt. The amorphous form provided herein is at least stable under ordinary stability conditions with respect to purity, storage and is a free flowing powder.

Amorphous solid dispersions of drugs are generally known to improve the stability and solubility of drug products. However, such dispersions are generally unstable over time. Amorphous solid dispersions of drugs tend to convert to crystalline forms over time, which can lead to improper dosing due to differences of the solubility of crystalline drug material compared to amorphous drug material.

The present invention also provides stable solid dispersions comprising amorphous Betrixaban and its maleate salt with improved solubility. Moreover, the present invention provides solid dispersions comprising amorphous Betrixaban and its maleate salt which may be reproduced easily and is amenable for processing into a dosage form.

### SUMMARY OF THE INVENTION

In the first embodiment, the present application provides Betrixaban maleate of Formula I′ in amorphous form.

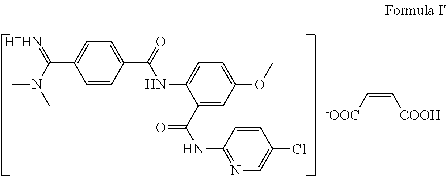

In the second embodiment, the present application provides amorphous Betrixaban maleate that can be characterized by its PXRD pattern as illustrated by FIG. 7.

In the third embodiment, the present application provides processes for preparing an amorphous form of Betrixaban maleate, comprising the steps of:

a) providing a solution of Betrixaban maleate in a solvent; and
b) isolating amorphous Betrixaban maleate.

In the fourth embodiment, the present application provides a pharmaceutical composition comprising an amorphous form of Betrixaban maleate and one or more pharmaceutically acceptable excipients.

In the fifth embodiment, the present application provides a solid dispersion comprising amorphous Betrixaban maleate and one or more pharmaceutically acceptable excipients.

In the sixth embodiment, the present application provides a method for preparing a solid dispersion comprising amorphous Betrixaban maleate and one or more pharmaceutically acceptable excipients comprising the steps of:

a) providing a mixture of Betrixaban maleate and one or more pharmaceutically acceptable polymers in a solvent; and
b) isolating solid dispersion comprising amorphous Betrixaban maleate and one or more pharmaceutically acceptable excipients.

In the seventh embodiment, the present application provides a method for preparing a solid dispersion comprising amorphous Betrixaban maleate and one or more pharmaceutically acceptable excipients comprising the steps of:

a) physically blending Betrixaban maleate and one or more pharmaceutically acceptable polymers; and
b) isolating solid dispersion comprising amorphous Betrixaban maleate and one or more pharmaceutically acceptable excipients.

In the eighth embodiment, the present application provides Betrixaban in amorphous form.

In the ninth embodiment, the present application provides amorphous Betrixaban that can be characterized by its PXRD pattern as illustrated by FIG. 3.

In the tenth embodiment, the present application provides process for preparing an amorphous form of Betrixaban, comprising the steps of:

a) providing a solution of Betrixaban in a solvent; and
b) isolating amorphous Betrixaban.

In the eleventh embodiment, the present application provides a pharmaceutical composition comprising an amorphous form of Betrixaban and one or more pharmaceutically acceptable excipients.

In the twelfth embodiment, the present application provides a solid dispersion comprising amorphous Betrixaban and one or more pharmaceutically acceptable polymers.

In the thirteenth embodiment, the present application provides a method for preparing a solid dispersion comprising amorphous Betrixaban and one or more pharmaceutically acceptable excipients comprising the steps of:

a) providing a mixture of Betrixaban and o one or more pharmaceutically acceptable excipients in a solvent; and
b) isolating solid dispersion comprising amorphous Betrixaban and one or more pharmaceutically acceptable excipients.

In the fourteenth embodiment, the present application provides a method for preparing a solid dispersion comprising amorphous Betrixaban and one or more pharmaceutically acceptable excipients comprising the steps of:

a) physically blending Betrixaban and one or more pharmaceutically acceptable excipients; and
b) isolating solid dispersion comprising amorphous Betrixaban and one or more pharmaceutically acceptable excipients.

In the fifteenth embodiment, the present application provides crystalline Form IV of Betrixaban maleate.

In the sixteenth embodiment, the present application provides crystalline Betrixaban maleate Forms IV which can be characterized by its PXRD pattern as illustrated by FIG. 15.

In the seventeenth embodiment, the present application provides processes for preparing crystalline Form IV of Betrixaban maleate, comprising the steps of:
a) providing a mixture of Betrixaban maleate in formic acid; optionally in presence of suitable solvent;
b) isolating the crystalline form of Betrixaban maleate.
   In the eighteenth embodiment, the present application provides crystalline Form V of Betrixaban maleate.

In the nineteen embodiment, the present application provides crystalline Betrixaban maleate Forms V which can be characterized by its PXRD pattern as illustrated by FIG. 16.

In the twentieth embodiment, the present application provides processes for preparing crystalline Form V of Betrixaban maleate, comprising the steps of:
a) providing a mixture of Betrixaban maleate in propylene glycol; optionally in presence of suitable solvent;
b) isolating the crystalline form of Betrixaban maleate.

### BRIEF DESCRIPTION OF THE DRAWING

FIG. 1 is an illustration of powder X-ray diffraction (“PXRD”) pattern of Betrixaban employed as an input material in examples 1-5.

FIG. 2 is an illustration of powder X-ray diffraction (“PXRD”) pattern of Betrixaban amorphous form prepared according to example 1.

FIG. 3 is an illustration of powder X-ray diffraction (“PXRD”) pattern of Betrixaban amorphous form prepared according to example 2.

FIG. 4 is an illustration of powder X-ray diffraction (“PXRD”) pattern of Betrixaban present in amorphous solid dispersion according to example 3.

FIG. 5 is an illustration of powder X-ray diffraction (“PXRD”) pattern of Betrixaban present in amorphous solid dispersion according to example 4.

FIG. 6 is an illustration of powder X-ray diffraction (“PXRD”) pattern of Betrixaban present in amorphous solid dispersion according to example 5.

FIG. 7 is an illustration of powder X-ray diffraction (“PXRD”) pattern of Betrixaban Maleate amorphous form prepared according to example 6.

FIG. 8 is an illustration of powder X-ray diffraction (“PXRD”) pattern of Betrixaban maleate amorphous form according to example 7.

FIG. 9 is an illustration of powder X-ray diffraction (“PXRD”) pattern of Betrixaban Maleate present in amorphous solid dispersion according to example 8.

FIG. 10 is an illustration of powder X-ray diffraction (“PXRD”) pattern of Betrixaban Maleate present in amorphous solid dispersion according to example 9.

FIG. 11 is an illustration of powder X-ray diffraction (“PXRD”) pattern of Betrixaban Maleate present in amorphous solid dispersion according to example 11.

FIG. 12 is an illustration of powder X-ray diffraction (“PXRD”) pattern of Betrixaban Maleate present in amorphous solid dispersion according to example 12.

FIG. 13 is an illustration of powder X-ray diffraction (“PXRD”) pattern of Betrixaban Maleate present in amorphous solid dispersion according to example 13.

FIG. 14 is an illustration of powder X-ray diffraction (“PXRD”) pattern of Betrixaban Maleate amorphous form according to example 18.

FIG. 15 is an illustration of powder X-ray diffraction (“PXRD”) pattern of Crystalline Form-IV of Betrixaban Maleate according to example 19.

FIG. 16 is an illustration of powder X-ray diffraction (“PXRD”) pattern of Crystalline Form-V of Betrixaban Maleate according to example 20.

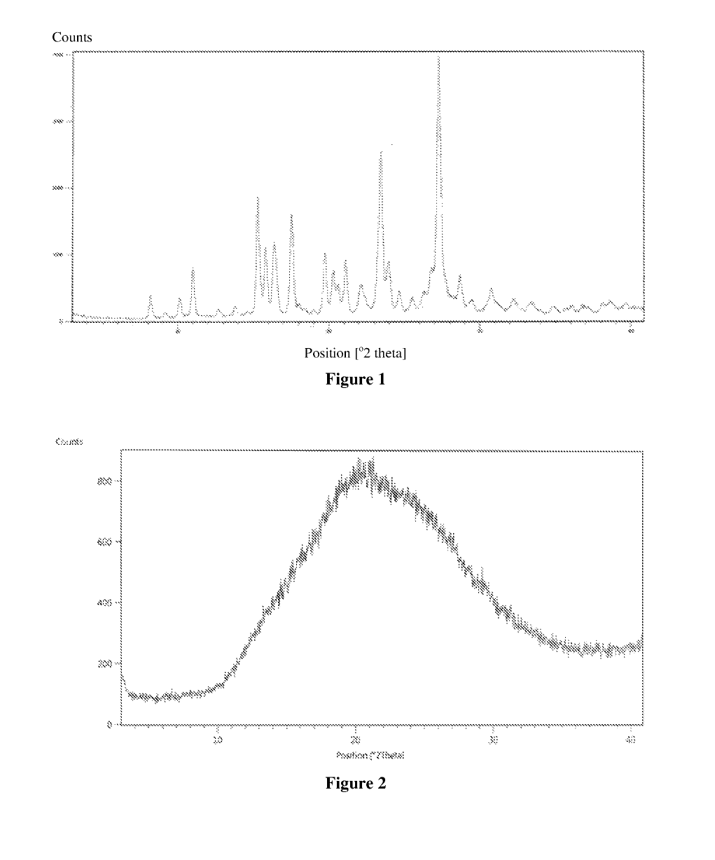

### DESCRIPTION OF THE INVENTION

In an embodiment, the present application provides processes for preparing an amorphous form of Betrixaban or its maleate salt, comprising the steps of:

a) providing a solution of Betrixaban or its maleate salt in a solvent; and
b) isolating amorphous Betrixaban or its maleate salt.
Providing a solution in step a) includes:
i) direct use of a reaction mixture containing Betrixaban or its maleate salt that is obtained in the course of its synthesis; or
ii) direct use of reaction mixture containing Betrixaban maleate that is obtained by treating Betrixaban with Maleic acid; or
ii) dissolving Betrixaban or its maleate salt in a solvent.

Any physical form of Betrixaban or its maleate salt may be utilized in step (a) of the process embodiments herein above.

Suitable solvents which can be used in step (a) for the preparation of amorphous Betrixaban or its maleate salt include but are not limited to: alcoholic solvents such as methanol, ethanol, isopropyl alcohol, n-propanol, isoamyl alcohol and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, di-isopropyl ether, 1,4-dioxane and the like; hydrocarbons such as toluene, xylene and the like; nitriles such as acetonitrile, propionitrile and the like; Organic acids such as Formic acid, Acetic acid and the like; solvents like DMSO, DMF, DMA and the like; water and any mixtures of two or more thereof.

The solution obtained in step (a) may be optionally filtered to remove any insoluble particles. Suitable techniques to remove insoluble particles are filtration, micron filter, centrifugation, decantation, and any other techniques known in the art. The solution can be filtered by passing through paper, glass fiber, or other membrane material, or a clarifying agent such as celite. Depending upon the equipment used and the concentration and temperature of the solution, the filtration apparatus may need to be preheated to avoid premature precipitation of solid.

Step (b) for involves isolating the amorphous Betrixaban or its maleate salt.

In one aspect present application involves isolation of amorphous Betrixaban or its maleate salt by removing solvent from a solution obtained in step (a). Suitable techniques which can be used for the removal of solvent include but not limited to evaporation, flash evaporation, simple evaporation, rotational drying, spray drying, thin-film drying (e.g., agitated thin-film drying (ATFD)), agitated nutsche filter drying, pressure nutsche filter drying, freeze-drying, rotary vacuum paddle dryer (RVPD) or any other suitable technique known in the art.

The solvent can be removed, optionally under reduced pressures, at temperatures less than about 100° C., less than about 75° C., less than about 60° C., less than about 50° C., less than about 40° C. or any other suitable temperatures.

In another aspect present application involves recovery of an amorphous form of Betrixaban or its maleate salt after removal of solvent. The said recovery can be done by using the processes known in the art. The resulting solid may be collected by using techniques such as by scraping, or by shaking the container, or other techniques specific to the equipment used. The isolated solid may be optionally further dried to afford an amorphous form of Betrixaban or its maleate salt.

The resulting compound may be optionally further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 100° C., less than about 70° C., less than about 40° C., less than about 30° C., less than about 20° C., or any other suitable temperatures; at atmospheric pressure or under a reduced pressure; as long as the Betrixaban or its maleate salt is not degraded in its quality. The drying can be carried out for any desired times until the required product quality is achieved. Suitable time for drying can vary from few minutes to several hours for example from about 30 minutes to about 24 or more hours.

Solid dispersion as used herein refers to the dispersion of one or more active ingredients in an inert excipient or polymer or carrier, where the active ingredients could exist in finely crystalline, solubilized or amorphous state (Sareen et al., 2012 and Kapoor et al., 2012). Solid dispersion consists of two or more components, generally an excipient or polymer or carrier and drug optionally along with stabilizing agent (and/or surfactant or other additives). The most important role of the added excipient/polymer/carrier in solid dispersion is to reduce the molecular mobility of the drug to avoid the phase separation and re-crystallization of drug during storage. The resulting solid dispersions may have increased solubility. The increase in solubility of the drug in solid dispersion is mainly because drug remains in amorphous form which is associated with a higher energy state as compared to crystalline counterpart and due to that it requires very less external energy to dissolve.

A solid dispersion is a molecular dispersion of a compound, particularly a drug substance within an excipient or polymer or carrier. Formation of a molecular dispersion provides a means of reducing the particle size to nearly molecular levels (i.e. there are no particles). As the polymer dissolves, the drug is exposed to the dissolution media as fine particles that are amorphous, which can dissolve and be absorbed more rapidly than larger particles.

In general, the term “solid dispersion” refers to a system in a solid state comprising at least two components, wherein one component is dispersed throughout the other component or components. The term “solid dispersion” as used herein, refers to stable solid dispersions comprising amorphous drug substance and one or more polymers or carriers. Further the term “solid dispersion” as used herein also refers to stable solid dispersions comprising amorphous drug substance and one or more excipients or polymers or carriers with or without adsorbent/absorbent. By “amorphous drug substance,” it is meant that the amorphous solid contains drug substance in a substantially amorphous solid state form i.e. at least about 80% of the drug substance in the dispersion is in an amorphous form. More preferably at least about 90% and most preferably at least about 95% of the drug substance in the dispersion is in amorphous form.

The solid dispersions of Betrixaban or its maleate salt of the present invention can be made by any of numerous methods that result in a solid dispersion comprising an amorphous Betrixaban or its maleate salt. Several approaches can be used for the preparation of solid dispersion which includes spray drying, fusion method, solvent evaporation, hot-melt extrusion, particle size reduction, supercritical fluid (SCF) processes, kneading, inclusion complexes, electrostatic spinning method, melt crystallization and surface-active carriers.

Betrixaban or its maleate salt can be incorporated in the dispersion is amorphous.

The dispersing agent is typically composed of a pharmaceutically acceptable substance that does not substantially interfere with the pharmaceutical action of Betrixaban or its maleate salt. The phrase “pharmaceutically acceptable” is employed herein to refer to those substances which are, within the scope of sound medical judgment, suitable for use in contact with the tissues of human beings and animals without excessive toxicity, irritation, allergic response, or other problem or complication, commensurate with a reasonable benefit/risk ratio. In some embodiments, the carrier is a solid at room temperature (e.g., about 22° C.).

In an embodiment suitable pharmaceutically acceptable excipients or polymers or carriers that are optionally dispersing agents which can be used for the preparation of solid dispersion include, but are not limited to: diluents such as starches, pregelatinized starches, lactose, powdered celluloses, microcrystalline celluloses, dicalcium phosphate, tricalcium phosphate, mannitol, sorbitol, sugar and the like; binders such as acacia, guar gum, tragacanth, gelatin, polyvinylpyrrolidones, hydroxypropyl celluloses, hydroxypropyl methylcelluloses, pregelatinized starches and the like; disintegrants such as starches, sodium starch glycolate, pregelatinized starches, crospovidones, croscarmellose sodium, colloidal silicon dioxide and the like; lubricants such as stearic acid, magnesium stearate, zinc stearate and the like; glidants such as colloidal silicon dioxide (Syloid, Aerosil, Cab-o-sil etc.) and the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, waxes and the like; celluloses (e.g., carboxymethylcelluloses, methylcelluloses, hydroxypropylcelluloses, hydroxypropylmethylcelluloses); polysaccharides, heteropolysaccharides (pectins); poloxamers; poloxamines; ethylene vinyl acetates; polyethylene glycols; dextrans; polyvinylpyrrolidones; chitosans; polyvinylalcohols; propylene glycols; polyvinylacetates; phosphatidylcholines (lecithins); miglyols; polylactic acid; polyhydroxybutyric acid; mixtures of two or more thereof, copolymers thereof, derivatives thereof, and the like. Further examples of carriers include copolymer systems such as polyethylene glycol-polylactic acid (PEG-PLA), polyethylene glycol-polyhydroxybutyric acid (PEG-PHB), polyvinylpyrrolidone-polyvinylalcohol (PVP-PVA), and derivatized copolymers such as copolymers of N-vinyl purine (or pyrimidine) derivatives and N-vinylpyrrolidone. Other pharmaceutically acceptable excipients that are of use include but are not limited to film formers, plasticizers, colorants, flavoring agents, sweeteners, viscosity enhancers, preservatives, antioxidants, and the like.

An enteric coating polymer can also be used according to the present invention. Specific examples of the enteric coating polymers include cellulose acetate phthalate, cellulose acetate trimellitate, cellulose acetate succinate, hydroxymethylcellulose ethyl phthalate, hydroxypropylmethylcellulose phthalate, eudragit, hydroxypropylmethylcellulose acetate succinate, hydroxypropylmethyl acetate maleate, hydroxypropylmethyl trimellitate, carboxymethylethylcellulose, polyvinyl butyrate phthalate, polyvinyl alcohol acetate phthalate, methacrylic acid/ethyl acrylate copolymer, and methacrylic acid/methyl methacrylate copolymer, hydroxypropyl methylcellulose phthalate, hydroxypropylmethylcellulose acetate succinate, hydroxypropylmethyl acetate maleate and hydroxypropylmethyl trimellitate.

In an aspect of the invention, the polymer is polyvinylpyrrolidone (PVP) or a derivative thereof. PVP is a polyamide that forms complexes with a wide variety of substances and is considered to be chemically and physiologically inert. Examples of suitable PVPs include polyvinylpyrrolidones having an average molecular weight from about 10,000 to about 50,000. In some embodiments, the polyvinylpyrrolidone has an average molecular weight of about 10,000 to about 20,000. In further embodiments, the polyvinylpyrrolidone has a molecular weight of about 15,000 to about 20,000.

In another embodiment, the present application provides a method for preparing a solid dispersion comprising amorphous Betrixaban or its maleate salt and one or more pharmaceutically acceptable excipients comprising the steps of:

a) providing a mixture of Betrixaban or its maleate salt and one or more pharmaceutically acceptable excipients in a solvent; and
b) isolating solid dispersion comprising amorphous Betrixaban or its maleate salt and one or more pharmaceutically acceptable excipients.

Any physical form of Betrixaban or its maleate salt may be utilized for providing the mixture of Betrixaban or its maleate salt in step (a).

In an aspect for the preparation of solid dispersion comprising amorphous Betrixaban or its maleate salt and one or more pharmaceutically acceptable polymers or carriers, step (a) involves mixing Betrixaban or its maleate salt and one or more pharmaceutically acceptable polymers or carriers for providing a solution or a suspension in step (a).

Suitable solvent which can be used for preparing the solid dispersion of Betrixaban or its maleate salt are the same as described herein above.

Any undissolved particles in the solution obtained in step (a) may be removed by suitable method as described herein above or any other technique known in the art.

The step (b) involves isolation of solid dispersion comprising amorphous Betrixaban or its maleate salt and one or more pharmaceutically acceptable polymers.

In one aspect, isolation of solid dispersion can be carried out by removing solvent from a solution obtained in step (a).

Suitable techniques which can be used for the removal of solvent are the same as described herein above or any other technique known in the art.

Although the solid dispersions of the present invention are preferably prepared using conventional spray drying techniques, it will be understood that suitable solid dispersions may be formed utilizing other conventional techniques known to those skilled in the art, such as vacuum drying, fluid-bed drying, freeze-drying, rotary evaporation, drum drying, or other solvent removal process.

Another aspect of the invention involves preparation of solid dispersions of Betrixaban or its maleate salt by melt processing, wherein the compound and a carrier are heated to a temperature above the melting point of both the carrier and compound, which results in the formation of a fine colloidal (as opposed to molecular) dispersion of compound particles, with some solubilization of the compound in the carrier matrix. Processing of such a molten mixture often includes rapid cooling, which results in the formation of a congealed mass which must be subsequently milled to produce a powder which can be filled into capsules or made into tablets.

In another aspect present application involves recovery of solid dispersion comprising an amorphous form of Betrixaban or its maleate and one or more pharmaceutically acceptable polymers or carriers. The said recovery can be carried out by methods as described herein above or any other technique known in the art.

The resulting solid dispersion comprising an amorphous form of Betrixaban or its maleate and one or more pharmaceutically acceptable polymers or carriers may be optionally further dried. Drying can be can be carried out by methods as described herein above or any other technique known in the art.

In an another aspect, present application provides a method for preparing a solid dispersion comprising amorphous Betrixaban or its maleate salt and one or more pharmaceutically acceptable excipients comprising the steps of:

a) physically blending of Betrixaban or its maleate salt and one or more pharmaceutically acceptable excipients; and
b) isolating solid dispersion comprising amorphous Betrixaban or its maleate salt and one or more pharmaceutically acceptable excipients.

Betrixaban or its maleate salt substantially in amorphous form may be utilized for physical blending of Betrixaban or its maleate salt in step (a).

Suitable pharmaceutically acceptable polymers or carriers that are dispersing agents which can be used in step (a) are the same as defined herein above.

Physical blending as used in step a) involves dry blending in motor pistol, flask or any other suitable container or any other technique known in the art.

Step (b) involves isolation of solid dispersion comprising amorphous Betrixaban or its maleate salt and one or more pharmaceutically acceptable excipients or polymers or carriers which can be carried out by any technique known in the art.

The amount of Betrixaban or its maleate salt in the solid dispersions of the present invention ranges from about 0.1% to about 90% by weight of the solid dispersion; or from about 10% to about 70% by weight of the solid dispersion; or from about 20% to about 60% by weight of the solid dispersion; or from about 20% to about 40% by weight of the solid dispersion; or about 30% by weight of the solid dispersion. In some aspects, the weight ratio of Betrixaban or its maleate salt to polymer or carrier is about 1:99 to about 99:1. In some aspects, the weight ratio of Betrixaban or its maleate salt to polymer or carrier is about 1:99 to about 75:25 or about 1:99 to about 60:40. In further aspects, the weight ratio of Betrixaban or its maleate salt to polymer or carrier is about 1:99 to about 15:85; about 1:99 to about 10:90; or about 1:99 to about 5:95. In further aspects, the weight ratio of Betrixaban or its maleate salt to polymer or carrier is about 25:75 to about 75:25, about 40:60 to about 60:40 or about 1:1 or about 2:1. Typically, Betrixaban or its maleate salt and carrier medium are present in a ratio by weight with the solvent of 1:0.1 to 1:20.

Amorphous forms or the solid dispersions of Betrixaban or its maleate salt of the present application can be optionally subjected to a particle size reduction procedures before or after the completion of drying of the product to produce desired particle sizes and distributions. Milling or micronization can be performed to achieve the desired particle sizes or distributions. Equipment that may be used for particle size reduction include, without limitation thereto, ball mills, roller mills, hammer mills, and jet mills.

In another general aspect, there is provided amorphous form of Betrixaban or its maleate salt or solid dispersion comprising amorphous form of Betrixaban or its maleate salt having particle size distributions wherein D90 is less than about 500 microns or less than about 200 microns or less than about 100 microns or less than about 50 microns or less than about 40 microns or less than about 30 microns or less than about 20 microns or less than about 10 microns or any other suitable particle sizes.

In an embodiment, the present application provides crystalline Form IV of Betrixaban maleate.

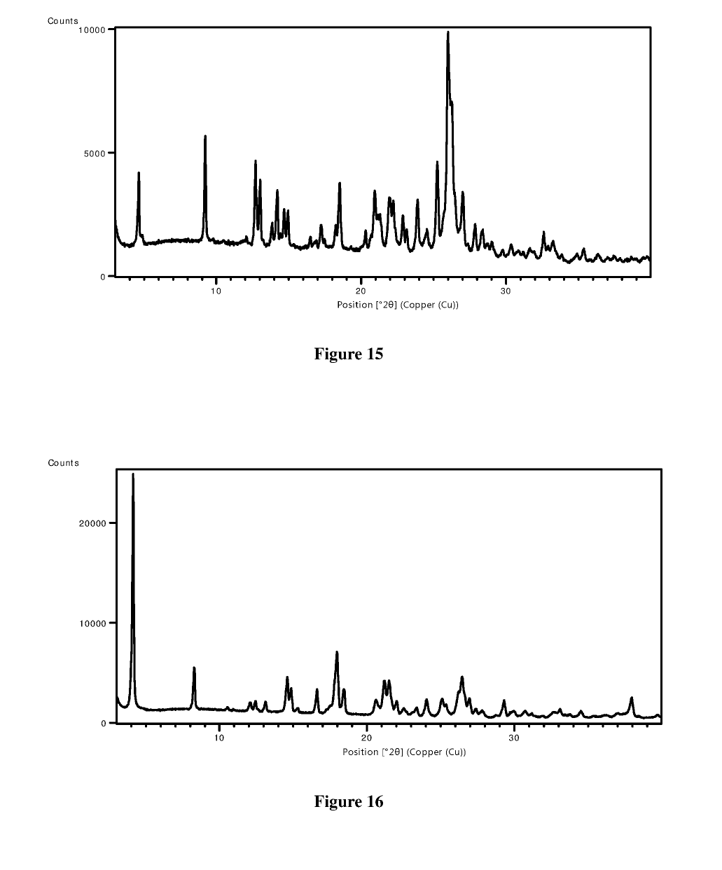

In another embodiment, the present application provides crystalline Betrixaban maleate Forms IV which can be characterized by its PXRD pattern as illustrated by FIG. 15.

In yet another aspect, the present invention relates to crystalline Form IV characterized by XRPD having the following approximate characteristic peak locations at 9.21, 12.69 and 13.00±0.2 theta.

In another aspect, the present invention relates to crystalline Form IV further characterized by XRPD having the following approximate characteristic peak locations at 14.17, 18.49, 20.91, 23.86 and 25.23±0.2 theta.

In yet another embodiment, the present application provides processes for preparing crystalline Form IV of Betrixaban maleate, comprising the steps of:
c) providing a mixture of Betrixaban maleate in formic acid; optionally in presence of suitable solvent;
d) isolating the crystalline form of Betrixaban maleate.
   In an embodiment, the present application provides crystalline Form V of Betrixaban maleate.

In another embodiment, the present application provides crystalline Betrixaban maleate Forms V which can be characterized by its PXRD pattern as illustrated by FIG. 16.

In yet another aspect, the present invention relates to crystalline Form V characterized by XRPD having the following approximate characteristic peak locations at 4.13 and 8.27±0.2 theta.

In another aspect, the present invention relates to crystalline Form V further characterized by XRPD having the following approximate characteristic peak locations at 14.58, 16.59, 17.92, 18.42, 21.16, 21.49, 24.03 and 26.45±0.2 theta.

In yet another embodiment, the present application provides processes for preparing crystalline Form V of Betrixaban maleate, comprising the steps of:
c) providing a mixture of Betrixaban maleate in propylene glycol; optionally in presence of suitable solvent;
d) isolating the crystalline form of Betrixaban maleate.

Any physical form of Betrixaban maleate may be utilized in step (a) of the process embodiments herein above.

Suitable solvents which can be used in step (a) for the preparation of crystalline Betrixaban maleate salt in Form IV or Form V include but are not limited to: ethers such as diethyl ether, methyl tert-butyl ether (MTBE), dimethyl ether, di-isopropyl ether, 1,4-dioxane and the like alcoholic solvents such as methanol, ethanol, isopropyl alcohol, n-propanol, isoamyl alcohol and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; hydrocarbons such as toluene, xylene and the like; nitriles such as acetonitrile, propionitrile and the like; solvents like DMSO, DMF, DMA and the like; water and any mixtures of two or more thereof. Suitable solvent or anti-solvent can be chosen from the above list by a skilled person.

If the mixture obtained in step (a) is solution then it may be optionally filtered to remove any insoluble particles. Suitable techniques to remove insoluble particles are filtration, micron filter, centrifugation, decantation, and any other techniques known in the art. The solution can be filtered by passing through paper, glass fiber, or other membrane material, or a clarifying agent such as celite. Depending upon the equipment used and the concentration and temperature of the solution, the filtration apparatus may need to be preheated to avoid premature precipitation of solid.

Step (b) involves isolating the crystalline Betrixaban maleate.

The isolation of crystalline Form IV or V of Betrixaban maleate may be induced by using conventional techniques known in the art. For example, useful techniques include but are not limited to, concentrating, cooling, stirring, shaking, combining with an anti-solvent, adding seed crystals, evaporation, flash evaporation, simple evaporation, rotational drying, spray drying, thin-film drying, freeze-drying, or the like. The solid that is obtained may carry a small proportion of occluded mother liquor containing a higher percentage of impurities and, if desired, the solid may be washed with a solvent to wash out the mother liquor. Evaporation as used herein refers to distilling of solvent almost completely at atmospheric pressure or under reduced pressure. Flash evaporation as used herein refers to distilling of solvent by using a technique includes but is not limited to tray drying, spray drying, fluidized bed drying, thin film drying under reduced pressure, or thin film drying at atmospheric pressure. The recovery of crystalline Form IV or V of Betrixaban maleate can be done by decantation, centrifugation, gravity filtration, suction filtration and like.

In a preferred embodiment, propylene glycol can be obtained in either enantioenriched (R) or (S) forms, or as an equal or unequal mixture of enantiomers.
In a preferred embodiment, anti-solvent is added for initiating crystallization. In yet another embodiment, methyl tert-butyl ether (MTBE) is employed as an anti-solvent.

Particularly, crystalline forms may also be obtained by heating or melting a form obtained followed by gradual or fast cooling; in this manner one polymorph or one crystalline form may be converted to another.

The solvent can be removed, optionally under reduced pressures, at temperatures less than about 100° C., less than about 75° C., less than about 60° C., less than about 50° C., less than about 40° C. or any other suitable temperatures.

In another aspect present application involves recovery of crystalline Betrixaban maleate salt after removal of solvent. The said recovery can be done by using the processes known in the art. The resulting solid may be collected by using techniques such as by scraping, or by shaking the container, or other techniques specific to the equipment used. The isolated solid may be optionally further dried to afford crystalline Betrixaban maleate.

The resulting compound may be optionally further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 100° C., less than about 70° C., less than about 40° C., less than about 30° C., less than about 20° C., or any other suitable temperatures; at atmospheric pressure or under a reduced pressure; as long as the crystalline Betrixaban maleate salt is not degraded in its quality. The drying can be carried out for any desired times until the required product quality is achieved. Suitable time for drying can vary from few minutes to several hours for example from about 30 minutes to about 24 or more hours.

Once obtained, crystals of Betrixaban maleate may be used as the nucleating agent or “seed” crystals for subsequent crystallizations of Betrixaban maleate from solutions.

In an aspect, the present application provides pharmaceutical formulations comprising an amorphous form of Betrixaban or its maleate salt or solid dispersion comprising amorphous form of Betrixaban or its maleate salt, or crystalline form of Betrixaban maleate together with one or more pharmaceutically acceptable excipients. Betrixaban or its maleate salt together with one or more pharmaceutically acceptable excipients of the present application may be formulated as: solid oral dosage forms such as, but not limited to, powders, granules, pellets, tablets, and capsules; liquid oral dosage forms such as, but not limited to, syrups, suspensions, dispersions, and emulsions; and injectable preparations such as, but not limited to, solutions, dispersions, and freeze dried compositions. Formulations may be in the forms of immediate release, delayed release, or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared using any one or more of techniques such as direct blending, dry granulation, wet granulation, and extrusion and spheronization. Compositions may be presented as uncoated, film coated, sugar coated, powder coated, enteric coated, and modified release coated.

Pharmaceutically acceptable excipients that are useful in the present application are the same as defined above.

The pharmaceutical dosage form according to the present invention may be is coated with one or more coating materials or uncoated. The coating materials are not particularly limited and are known to the person skilled in the art.

The pharmaceutical dosage form according to the present invention can further comprise additional excipients and adjuvants, which are pharmaceutically acceptable and general coating materials, which are preferably applied as a coating to the pharmaceutical dosage form of the present invention. Such further excipients and adjuvants are known to the person skilled in the art.

The pharmaceutical compositions of the present invention are generally administered orally to patients, which include, but are not limited to, mammals, for example, humans, in the form of, for example, a hard or soft gelatin capsule, a tablet, a caplet, pills, granules or a suspension. The pharmaceutical dosage form can be prepared by methods known in the art, such as direct compression or wet granulation or direct compression. The compression of the blend to tablet cores can be carried out using a conventional tableting machine or a rotary compression machine. The tablet cores may vary in shape and can be, for example, round, oval, oblong, cylindrical or any other suitable shape. The cores may also vary in size depending on the concentration of the therapeutic agent.

Betrixaban or its maleate salt which may be used as the input in the process for preparation of the solid states of the present application can be prepared by any process known in the art.

The solid form of Betrixaban or its maleate salt of the present application may be characterized by means of Powder X-ray Diffraction Pattern (PXRD). Other techniques, such as solid state NMR, Fourier Transform Infrared (FTIR), differential scanning calorimetry (DSC) may also be used.

The compound of this application is best characterized by the X-ray powder diffraction pattern determined in accordance with procedures that are known in the art.

PXRD data reported herein was obtained using CuKα radiation, having the wavelength 1.5406 Å and were obtained using a Bruker AXS D8 Advance Powder X-ray Diffractometer and PANalytical X'Pert PRO instruments. For a discussion of these techniques see J. Haleblain, J. Pharm. Sci. 1975 64:1269-1288, and J. Haleblain and W. McCrone, J. Pharm. Sci. 1969 58:911-929.

Generally, a diffraction angle (2θ) in powder X-ray diffractometry may have an error in the range of ±0.2°. Therefore, the aforementioned diffraction angle values should be understood as including values in the range of about ±0.2°. Accordingly, the present application includes not only crystals whose peak diffraction angles in powder X-ray diffractometry completely coincide with each other, but also crystals whose peak diffraction angles coincide with each other with an error of about ±0.2°. Therefore, in the present specification, the phrase “having a diffraction peak at a diffraction angle (2θ±0.2°) of 7.9°” means “having a diffraction peak at a diffraction angle (2 θ) of 7.7° to 8.1°”. Although the intensities of peaks in the x-ray powder diffraction patterns of different batches of a compound may vary slightly, the peaks and the peak locations are characteristic for a specific polymorphic form. Alternatively, the term “about” means within an acceptable standard error of the mean, when considered by one of ordinary skill in the art. The relative intensities of the PXRD peaks can vary depending on the sample preparation technique, crystal size distribution, various filters used, the sample mounting procedure, and the particular instrument employed. Moreover, instrument variation and other factors can affect the 2-theta values. Therefore, the term “substantially” in the context of PXRD is meant to encompass that peak assignments can vary by plus or minus about 0.2 degree. Moreover, new peaks may be observed or existing peaks may disappear, depending on the type of the machine or the settings (for example, whether a Ni filter is used or not).

The D10, D50, and D90 values are useful ways for indicating a particle size distribution. D90 refers to at least 90 volume percent of the particles having a size smaller than the said value. Likewise, D10 refers to 10 volume percent of the particles having a size smaller than the said value. D50 refers to 50 volume percent of the particles having a size smaller than the said value. Methods for determining D10, D50, and D90 include laser diffraction, such as using equipment from Malvern Instruments Ltd. of Malvern, Worcestershire, United Kingdom.

Certain specific aspects and embodiments of the present application will be explained in greater detail with reference to the following examples, which are provided by way of illustration only and should not be construed as limiting the scope of the application in any manner.

### Definitions

The following definitions are used in connection with the present application unless the context indicates otherwise. Polymorphs are different solids sharing the same molecular formula, yet having distinct physical properties when compared to other polymorphs of the same formula. The abbreviation “MC” mean moisture content. Moisture content can be conveniently measured, for example, by the Karl Fischer method.

“Amorphous form” as used herein refers to a solid state wherein the amorphous content with in the said solid state is at least about 35% or at least about 40% or at least about 45% or at least about 50% or at least about 55% or at least about 60% or at least about 65% or at least about 70% or at least about 75% or at least about 80% or at least about 85% or at least about 90% or at least about 95% or at least about 96% or at least about 97% or at least about 98% or at least about 99% or about 100%.

“Crystalline form” as used herein refers to a solid state wherein the crystalline content with in the said solid state is at least about 35% or at least about 40% or at least about 45% or at least about 50% or at least about 55% or at least about 60% or at least about 65% or at least about 70% or at least about 75% or at least about 80% or at least about 85% or at least about 90% or at least about 95% or at least about 96% or at least about 97% or at least about 98% or at least about 99% or about 100%.

All percentages and ratios used herein are by weight of the total composition, unless the context indicates otherwise. All temperatures are in degrees Celsius unless specified otherwise and all measurements are made at 25° C. and normal pressure unless otherwise designated. The present disclosure can comprise the components discussed in the present disclosure as well as other ingredients or elements described herein.

As used herein, “comprising” means the elements recited, or their equivalents in structure or function, plus any other element or elements which are not recited. The terms “having” and “including” are also to be construed as open ended unless the context suggests otherwise.

All ranges recited herein include the endpoints, including those that recite a range “between” two values.

Terms such as “about,” “generally,” “substantially,” or the like are to be construed as modifying a term or value such that it is not an absolute. Such terms will be defined by the circumstances and the terms that they modify, as those terms are understood by those of skill in the art. This includes, at very least, the degree of expected experimental error, technique error and instrument error for a given technique used to measure a value.

Where this document refers to a material, such as in this instance, Betrixaban or its maleate salt, and their solid state forms thereof by reference to patterns, spectra or other graphical data, it may do so by qualifying that they are “substantially” shown or as depicted in a Figure, or by one or more data points. By “substantially” used in such a context, it will be appreciated that patterns, spectra and other graphical data can be shifted in their positions, relative intensities and/or values due to a number of factors known to those of skill in the art.

In addition, where a reference is made to a figure, it is permissible to, and this document includes and contemplates, the selection of any number of data points illustrated in the figure which uniquely define that solid state form, within any associated and recited margin of error, for purposes of identification.

When a molecule or other material is identified herein as “pure”, it generally means, unless specified otherwise, that the material is 99% pure or more, as determined by methods conventional in art such as high performance liquid chromatography (HPLC) or optical methods. In general, this refers to purity with regard to unwanted residual solvents, reaction byproducts, impurities, and unreacted starting materials. In the case of stereoisomers, “pure” also means 99% of one enantiomer or diastereomer, as appropriate. “Substantially” pure means, the same as “pure except that the lower limit is about 98% pure or more and likewise, “essentially” pure means the same as “pure” except that the lower limit is about 95% pure.

As used herein, the term “room temperature” refers to a temperature of from about 20° C. to about 35° C., from about 25° C. to about 35° C., from about 25° C. to about 30° C., or for example, about 25° C.

As used herein, the term “overnight” refers to a time interval from about 14 hours to about 24 hours, or about 14 hours to about 20 hours, for example, about 16 hours.

The “polymer” or “carrier” or “excipient” as used herein interchangeably refer to any substance or mixture of substances which are pharmaceutically acceptable inactive ingredients.

The term “dispersed” means random distribution of a therapeutically active substance throughout the carrier.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example 1: Preparation of Amorphous Betrixaban

Betrixaban (500 mg) and dichloromethane (150 mL) were charged into a flask and sonicated at room temperature for clear solution and filtered to remove undissolved particles. The resultant solution was subjected to fast solvent evaporation using rotavapor under 90 torr vacuum pressure at about 45° C. to afford the solid compound. The said solid was dried under vacuum at about 45° C. for about 2.5 hours to afford the amorphous Betrixaban according to FIG. 2.

### Example 2: Preparation of Amorphous Betrixaban

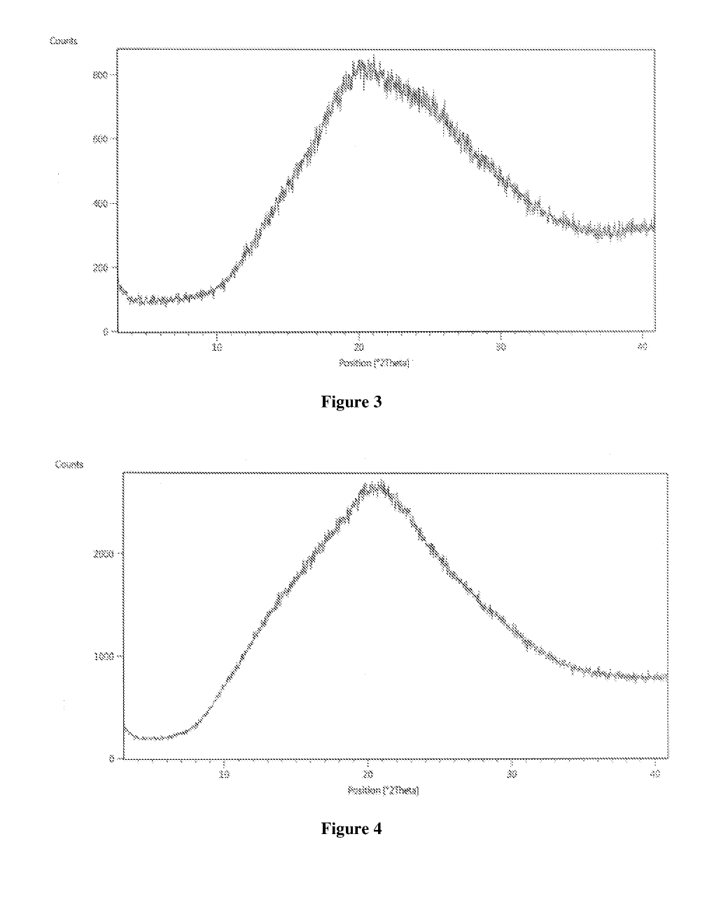

Betrixaban (4 g) was dissolved in a mixture of dichloromethane (475 mL) and methanol (205 mL). The solution is filtered to remove undissolved particles and the filtrate was subjected to spray drying at inlet temperature of 70° C. and outlet temperature of 46° C. with a flow rate of 15 mL/min to afford the title compound according to FIG. 3.

### Example 3: Preparation of Betrixaban Solid Dispersion with PVPK-30 (1:1 w/w)

To a solution of Betrixaban (500 mg) in dichloromethane (150 mL), polyvinylpyrrolidone (500 mg) was added. The mixture was stirred for clear solution and then subjected to solvent evaporation under 80 torr at about 45° C. to afford the solid. The said solid was dried under vacuum at 45° C. for about an hour.

The resulting dispersion was found to be amorphous by X-ray powder diffraction according to FIG. 4.

### Example 4: Preparation of Betrixaban Solid Dispersion with Co-Povidone (1:1 w/w)

To a solution of Betrixaban (500 mg) in dichloromethane (150 mL), Co-povidone (500 mg) was added. The mixture was stirred for clear solution and then subjected to fast solvent evaporation under 3 torr vacuum pressure at about 45° C. to afford the solid. The said solid was dried under vacuum at 3 torr pressure at about 45° C. for about 2.5 hours. The resulting solid dispersion was found to be amorphous by X-ray powder diffraction according to FIG. 5.

### Example 5: Preparation of Betrixaban Solid Dispersion with Co-Povidone and Syloid (1:1:1 w/w/w)

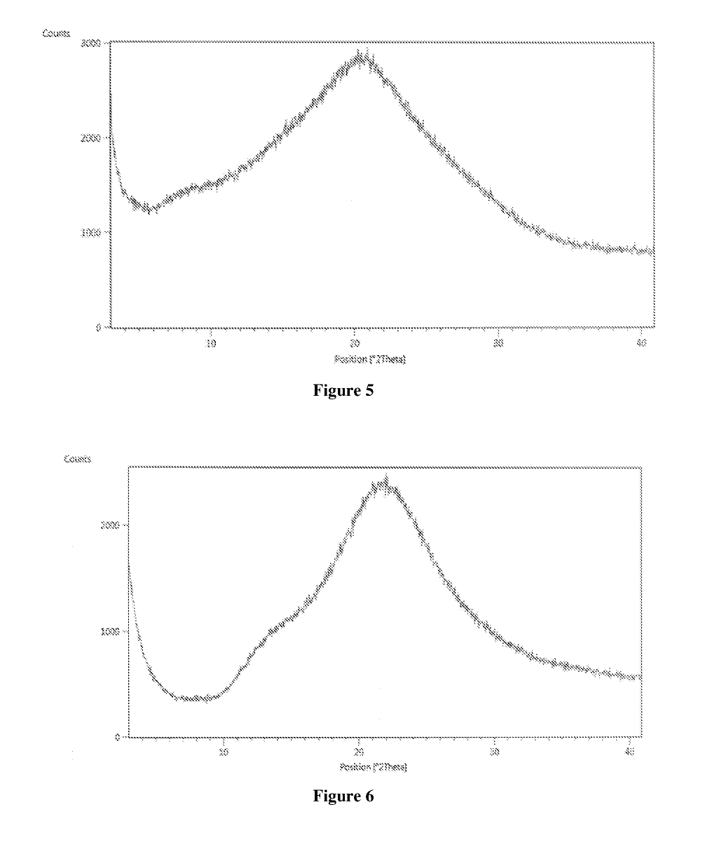

The solid dispersion (500 mg) prepared under example 4 was blended with Syloid (500 mg). The said blend was homogeneously mixed with grinding to afford the title solid dispersion. The resulting dispersion was found to be amorphous by X-ray powder diffraction according to FIG. 6.

### Example 6: Preparation of Amorphous Betrixaban Maleate

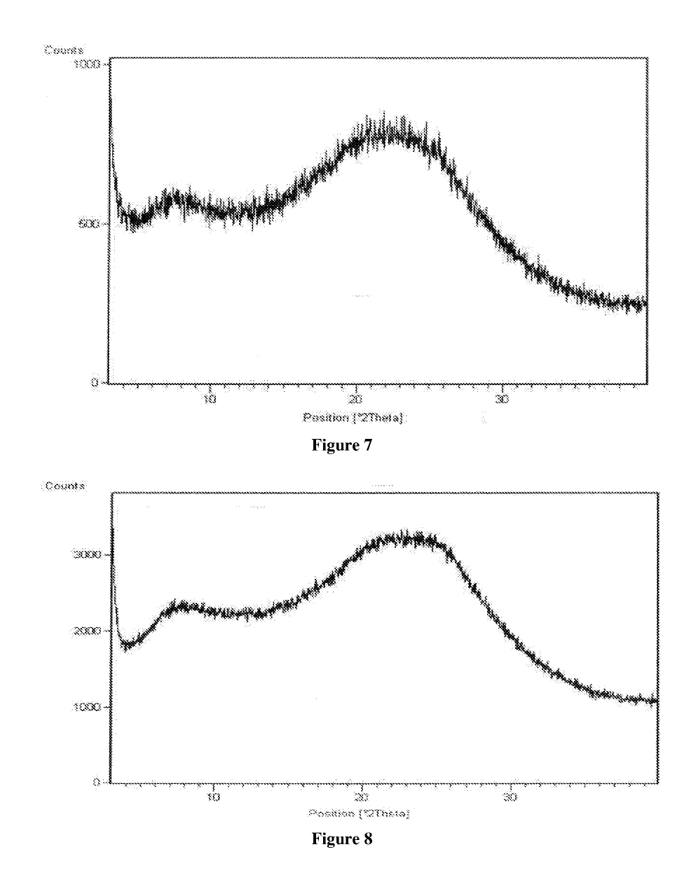

Betrixaban maleate (4 g) was dissolved in methanol (160 mL). The solution was sonicated and filtered to remove undissolved particles. The filtrate was subjected to spray drying at inlet temperature of 70° C., outlet temperature of 40° C. and aspirator 70% with a flow rate of 6 mL/min to afford the title compound according to FIG. 7.

### Example 7: Preparation of Amorphous Betrixaban Maleate

Betrixaban maleate (500 mg) was dissolved in methanol (15 mL) at about 60° C. The solution was subjected to fast solvent evaporation using rotavapor under 1 torr vacuum pressure at about 70° C. to afford the amorphous Betrixaban maleate according to FIG. 8.

### Example 8: Preparation of Amorphous Betrixaban Maleate Solid Dispersion with PVPK30 (1:1 w/w)

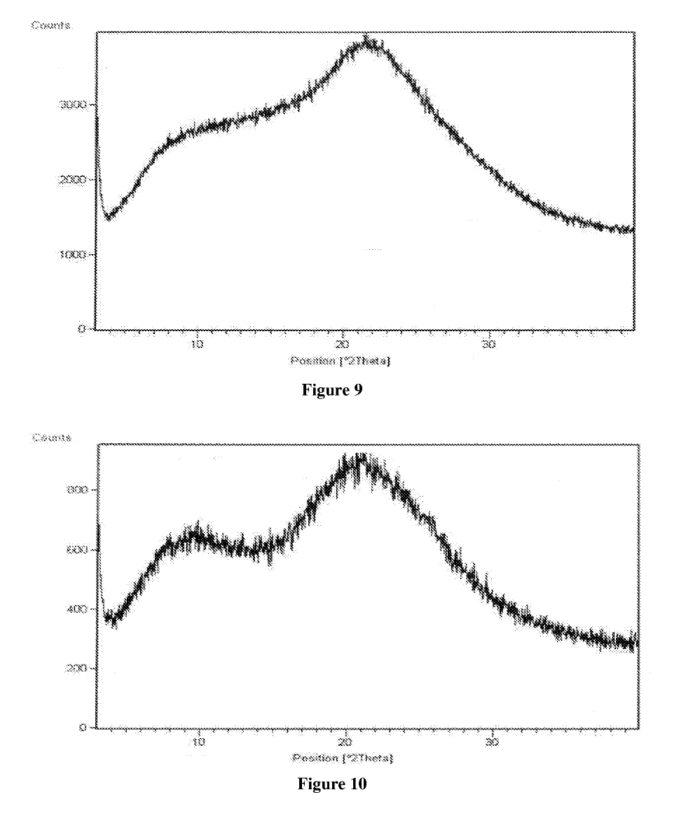

Betrixaban maleate (250 mg) and PVPK 30 (250 mg) were dissolved in methanol (12 mL) at room temperature. The mixture was filtered to remove undissolved particles and then subjected to solvent evaporation under 1 torr vacuum pressure at about 65° C. to afford the title solid. The resulting dispersion was found to be amorphous by X-ray powder diffraction according to FIG. 9.

### Example 9: Preparation of Amorphous Betrixaban Maleate Solid Dispersion with HPMC-AS (1:1 w/w)

Betrixaban maleate (250 mg) and HPMC-AS (250 mg) were dissolved in methanol (15 mL) at room temperature. The mixture was filtered to remove undissolved particles and then was subjected to fast solvent evaporation under 1 torr vacuum pressure at about 65° C. to afford the title solid. The resulting dispersion was found to be amorphous by X-ray powder diffraction according to FIG. 10.

### Example 10: Preparation of Amorphous Betrixaban Maleate Solid Dispersion with PVPK30 and Syloid (1:1:1 w/w/w)

Betrixaban maleate and PVPK30 amorphous solid dispersion/premix (200 mg) and Syloid-244 (100 mg) were ground into mortar-pestle for about 5 minutes to afford the title compound.

### Example 11: Preparation of Amorphous Betrixaban Maleate Solid Dispersion with PVPK30 and Syloid (1:1 w/w)

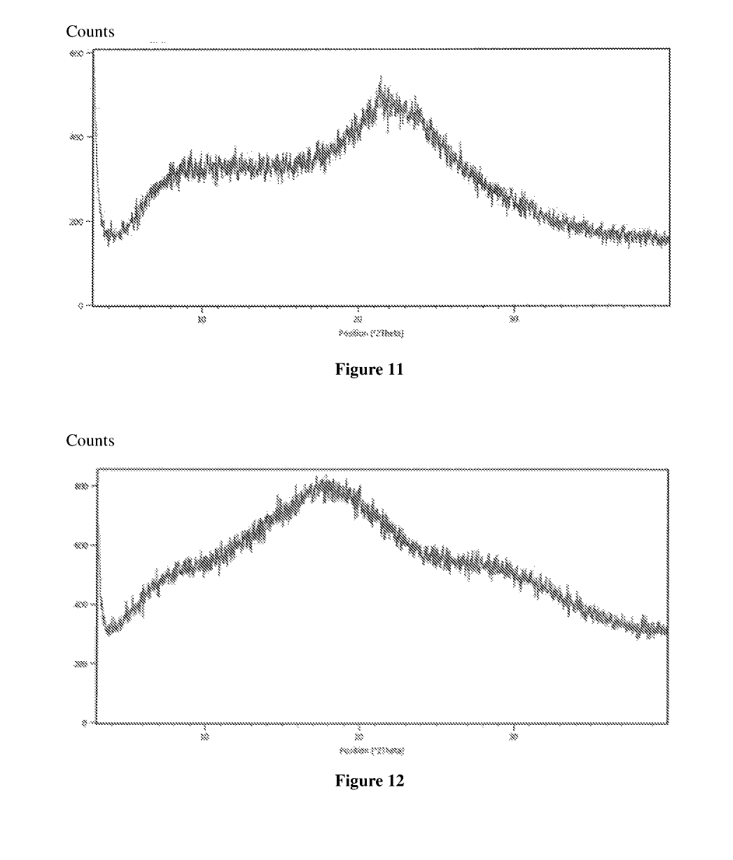

Betrixaban maleate and PVPK30 amorphous solid dispersion/premix (200 mg) and Syloid-244 (100 mg) were taken in methanol (12 mL) at room temperature. The resultant cloudy solution was subjected to fast solvent evaporation under 10 mbar vacuum pressure at about 70° C. to afford the title solid. The resulting solid dispersion was found to be amorphous by X-ray powder diffraction according to FIG. 11.

### Example 12: Preparation of Amorphous Betrixaban Maleate Solid Dispersion with Eudragit (1:1 w/w)

Betrixaban maleate (400 mg) and Eudragit (400 mg) were dissolved in methanol (25 mL) at room temperature. The mixture was filtered to remove undissolved particles and then was subjected to solvent evaporation under 1 torr at about 65° C. to afford the title solid. The resulting dispersion was found to be amorphous by X-ray powder diffraction according to FIG. 12.

### Example 13: Preparation of Amorphous Betrixaban Maleate Solid Dispersion with HPC (1:1 w/w)

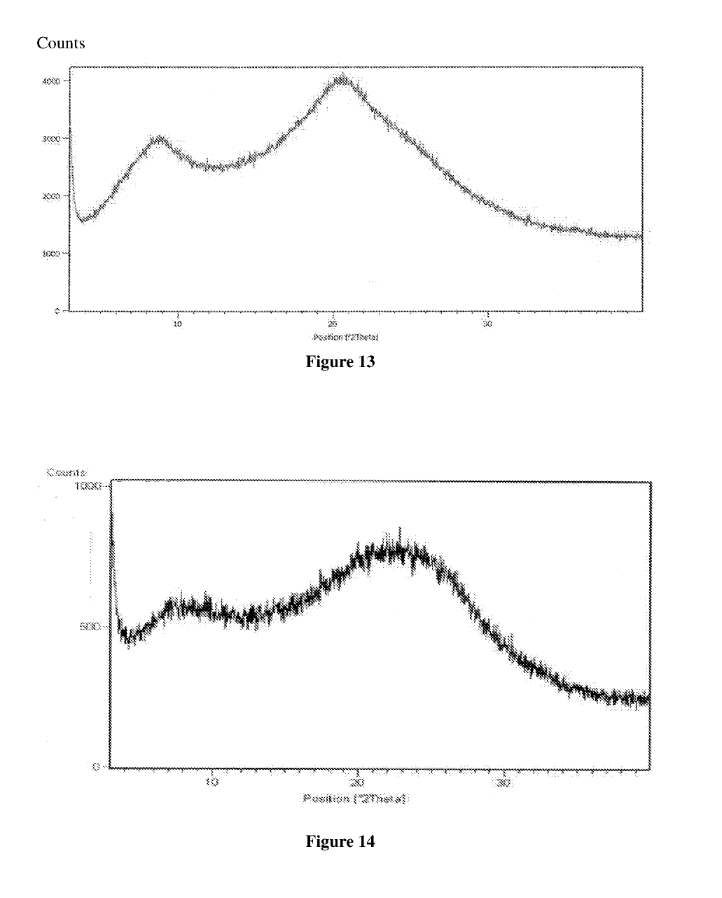

Betrixaban maleate (400 mg) and HPC-L (400 mg) were dissolved in methanol (25 mL) at room temperature. The mixture was filtered to remove undissolved particles and then was subjected to solvent evaporation under 1 torr at about 65° C. to afford the title solid. The resulting dispersion was found to be amorphous by X-ray powder diffraction according to FIG. 13.

### Example 14: Preparation of Amorphous Betrixaban Maleate Solid Dispersion with CPC and Syloid (1:1:1 w/w/w)

Betrixaban maleate and HPC amorphous solid dispersion/premix (200 mg) and Syloid-244 (100 mg) were ground into mortar-pestle for about 5 minutes to afford the title compound.

### Example 15: Preparation of Betrixaban Maleate Solid Dispersion with Eudragit and Syloid (1:1:1 w/w/w)

Betrixaban maleate and Eudragit amorphous solid dispersion/premix (200 mg) and Syloid-244 (100 mg) were ground into mortar-pestle for about 5 minutes to afford the title compound.

### Example 16: Preparation of Betrixaban Maleate Solid Dispersion with HPMC-AS and Syloid (1:1:1 w/w/w)

Betrixaban maleate and HPMC-AS amorphous solid dispersion/premix (200 mg) and Syloid-244 (100 mg) were ground into mortar-pestle for about 5 minutes to afford the title compound.

### Example 17: Preparation of Betrixaban Maleate Solid Dispersion with Syloid (1:1 w/w)

Amorphous Betrixaban maleate (150 mg) and Syloid-244 (150 mg) were ground into mortar-pestle for about 5 minutes to afford the title compound.

### Example 18: Preparation of Amorphous Betrixaban Maleate

Betrixaban maleate (1 g) was subjected to ball milling for 3 hours at 400 RPM with an interval of 10 minutes at about 30° C. to afford title compound according to FIG. 14.

### Example 19: Preparation of Crystalline Betrixaban Maleate (Form IV)

Betrixaban maleate (1 g) was dissolved in formic acid (2 mL) at ˜70° C. to obtain clear solution. The mixture was brought to room temperature followed by addition of MTBE (10 mL). The mixture was stirred at room temperature for about 2 hours and the solid is isolated by filtration and then dried to afford the title compound.

### Example 20: Preparation of Crystalline Betrixaban Maleate (Form V)

To a flask containing Betrixaban maleate (0.5 g), propylene glycol (2 mL) was added at room temperature. The mixture was stirred for about 3 hours followed by filtration of the obtained solid. The solid was washed with MTBE (2 mL) followed by its drying to afford the title compound.

1) Betrixaban Maleate of Formula I′ in solid amorphous form

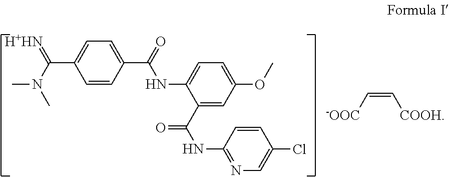

2) A process for preparing amorphous form of Betrixaban Maleate comprising:
a) providing a solution of Betrixaban maleate in a solvent, and
b) isolating amorphous Betrixaban maleate.

3) The process of claim 2 wherein suitable solvent in step a) is selected from alcohols, esters, ketones, hydrocarbons, water or mixtures thereof.

4) A process for preparation of solid dispersion comprising amorphous Betrixaban maleate, comprising:
a) providing a mixture of Betrixaban maleate and one or more pharmaceutically acceptable excipients in a solvent; and
b) isolating solid dispersion comprising amorphous Betrixaban maleate and one or more pharmaceutically acceptable excipients.

5) A solid dispersion comprising amorphous Betrixaban maleate and one or more pharmaceutically acceptable excipients.

6) The solid dispersion of claim 5, wherein the pharmaceutically acceptable excipients comprises of hydroxypropyl methyl cellulose (HPMC), hydroxypropyl cellulose (HPC), Polyvinyl pyrrolidone (PVP), silicon dioxide, copovidone, syloid, or suitable mixture thereof.

7) A process for preparation of solid dispersion comprising amorphous Betrixaban Maleate of claim 5 and one or more pharmaceutically acceptable excipients, comprising:
a) providing a mixture of Betrixaban maleate and one or more pharmaceutically acceptable excipients in a solvent; and
b) isolating solid dispersion comprising amorphous Betrixaban maleate and one or more pharmaceutically acceptable excipients.

8) A process for preparation of solid dispersion comprising amorphous Betrixaban Maleate of claim 5, comprising:
a) physically blending of Betrixaban maleate and one or more pharmaceutically acceptable excipients; and
b) isolating solid dispersion comprising amorphous Betrixaban maleate and one or more pharmaceutically acceptable excipients.

9) The physical blending as used in step a) of claim 8 involves dry blending in motor pistol, flask or any other suitable container or any other conventional technique.

10) A process for preparation of an amorphous form of Betrixaban, comprising the steps of:
a) providing a solution of Betrixaban in a solvent; and
b) isolating amorphous Betrixaban.

11) A crystalline Form-IV of Betrixaban maleate having an X-ray powder diffraction pattern having at least the following characteristic peak locations at 9.21, 12.69 and 13.00±0.2 theta or X-ray powder diffraction pattern shown in FIG. 15.

12) A crystalline Form-V of Betrixaban maleate having an X-ray powder diffraction pattern having at least the following characteristic peak locations at 4.13 and 8.27±0.2 theta or X-ray powder diffraction pattern shown in FIG. 16.

13) The crystalline Form V of Betrixaban maleate as defined in claim 12, further characterized by XRPD having the following approximate characteristic peak locations at 14.58, 16.59, 17.92, 18.42, 21.16, 21.49, 24.03 and 26.45±0.2 theta

14) A process for preparing crystalline Form IV of Betrixaban maleate, comprising the steps of:
a) providing a mixture of Betrixaban maleate in formic acid; optionally in presence of suitable solvent;
b) isolating the crystalline form of Betrixaban maleate.

15) A process for preparing crystalline Form V of Betrixaban maleate, comprising the steps of:
a) providing a mixture of Betrixaban maleate in propylene glycol; optionally in presence of suitable solvent;
b) isolating the crystalline form of Betrixaban maleate.
